(19) United States
(12) Patent Application Publication
(10) Pub. No.: US 2015/0083140 A1
(43) Pub. Date: Mar. 26, 2015

(54) MANDIBULOMAXILLARY STABILIZATION SYSTEM

(71) Applicants: Clemson University, Clemson, SC (US); Greenville Hospital Systems, Greenville, SC (US)

(72) Inventors: Tyler Youngman, Southlake, TX (US); Natalie Patzin, Naperville, IL (US); John O'Donnell, Gaithersburg, MS (US); Jeff Willis, Canastota, NY (US); Robert Brown, Greenville, SC (US)

(73) Assignee: Clemson University

(21) Appl. No.: 14/496,015

(22) Filed: Sep. 25, 2014

Related U.S. Application Data
(60) Provisional application No. 61/882,208, filed on Sep. 25, 2013.

(51) Int. Cl. A61B 17/80; B29C 67/00; B29C 51/10; A61C 7/08; A61C 7/36

(52) U.S. Cl. CPC A61B 17/8071; A61C 7/08; A61C 7/36; B29C 51/10; B29C 67/0051; A61B 2017/00955
USPC 128/862; 128/861; 264/16

(57) ABSTRACT

A mandibuiomaxillary fracture stabilization system, methods of forming the system, and methods of using the system are described. The system includes upper and lower splints that can be custom molded to fit the teeth of a patient and a locking mechanism that can temporarily lock the splints to one another. The splints are attached to the patient's dentition with a dental adhesive and the locking mechanism includes first and second components that are attached to the upper and lower splints, respectively. The locking mechanism includes a quick-release pin that allows the jaw to be freed in case of emergency.

### CROSS REFERENCE TO RELATED APPLICATION

This application claims filing benefit of U.S. Provisional Patent Application Ser. No. 61/882,208, having a filing date of Sep. 25, 2013, which is incorporated herein by reference in its entirety,

### BACKGROUND

Of the 206 bones in the human body, the mandible is the 10th most common fractured bone, and it is the 2nd most fractured bone in the facial skeleton for adults. Over three million facial fracture injuries occur in the United States annually, nearly one million of which are mandible and/or maxilla fractures that require reconstruction by specialized otolaryngologists or oral surgeons. This surgical reconstruction corrects the alignment of bones that have fractured after major trauma, tumor removal, deep facial tissue repair, plastic surgery, etc. The primary goal of the surgical process is keeping the maxilla and mandible properly aligned throughout surgery and the recovery period—insuring that the patient's natural bite is unaltered.

Currently, proper alignment is maintained during the recovery process through the use of an arch bar system illustrated in FIG. 1. Arch bars have been used with little change since the early 1900s to stabilize fractures of the mandible and maxilla. As shown in FIG. 1, an arch bar is fixed to both the upper and lower dentition by threading wire circumferentially around each tooth (FIG. 1A, FIG. 1B, FIG. 1C). The top and bottom arches are then securely wired shut by looping them together (FIG. 10). The entire mechanism remains in place, immobilizing the jaw for the six-week recovery period (FIG. 1E).

Despite the frequent use of the arch bar, there are many problems associated with it. Initially, installation is a highly complex and time-consuming process, taking anywhere from 45 minutes to 1 hour to install. Since operating rooms bill by the minute, decreasing this installation time is a way to significantly cut cost of a procedure. Moreover, the metal wire construction making up the arch bar can be very uncomfortable and irritating to the patient during the long recovery period and often causes soft tissue damage. The relatively permanent nature of the system also prevents quick removal in case of an emergency. Additionally, during installation, the sharp wires put the surgeon at risk of exposure to blood borne pathogens while threading and tightening the wires in the confined space of the mouth.

Attempts have been made to improve the arch bar system. For instance, an arch bar containing an upper and lower “U”-shaped bar has been developed that includes a splint portion that hides wires beneath a smooth rounded shelf. This arch bar limits soft tissue damage and reduces irritation. In another system, a fixation belt is connected to the mandible and maxilla by a plurality of connectors to secure both bones. This fixation belt contains a release mechanism that may be used in the event of an emergency with a push of a fingernail, Another system utilizes two brackets, two connectors, and one linkage device. This design allows for each bracket to be bonded to a single tooth with a dental adhesive, The assembly calls for two brackets to be applied to two teeth, one on the bottom and one on the top, the brackets are then linked to immobilize the mandible.

While the above describe improvement in the art, room for further improvement exists. What is needed in the art is a mandibulomaxillary stabilization system that can be quickly and easily formed and applied for temporary fixation of the temporomandibular joint.

### SUMMARY

According to one embodiment, disclosed is a mandibulomaxillary stabilization system. The system can likewise be considered an external skeletal fixation system or device, an external fixation system or device, or an external skeletal fixator. The system includes an upper splint, a lower splint, and a locking mechanism. The upper and lower splints are removably attachable to the upper and lower dentition, respectively. The splints are formed of a polymeric material. The lower splint includes impressions for at least the right and left lower posterior teeth and the upper splint includes impressions for at least the right and left upper posterior teeth. The locking mechanism can include a first component that is associated with the upper splint and a second component that is associated with the lower splint. The first and second components releasably interlock with one another, for instance in a hinge-type relationship, to hold the upper and lower splints in a fixed relationship to one another when in the locked orientation.

A method for stabilizing a temporomandibular joint is also disclosed. For example, a method can include adhering a first polymeric splint to the upper dentition of a subject, adhering a second polymeric splint to the lower dentition of a subject, and locking a first component and a second component of a locking mechanism to one another. The first and second components of the locking mechanism are associated with the first and second polymeric splints such that upon locking the components together, the joint is secured in a fixed orientation.

Also disclosed is a method for forming a fixation system. A method can include molding a first splint against a mold of the upper dentition of a patient, molding a second splint against a mold of the lower dentition of a patient, associating a first component of a locking mechanism with the first splint, and associating a second component of the locking mechanism with the second splint.

### BRIEF DESCRIPTION OF THE DRAWINGS

A full and enabling disclosure, including the best mode thereof, to one of ordinary skill in the art, is set forth more particularly in the remainder of the specification, including reference to the accompanying Figures, in which:

FIG. 1 illustrates the attachment of an arch bar to the teeth (FIG. 1A, FIG. 1B, FIG. 1C), the attachment of an upper and lower arch bar to one another (FIG. 1D) and a fully attached arch bar system (FIG. 1E) as known in the past.

FIG. 2 is a computerized tomography (CT) scan image as may be utilized to form a mandibulomaxillary stabilization system as disclosed herein.

FIG. 3 illustrates one embodiment of a splint of a system as disclosed herein.

FIG. 4 illustrates one embodiment of a locking mechanism for a system as disclosed herein.

FIG. 5 illustrates one embodiment of a stabilization system as disclosed herein.

FIG. 6 illustrates a stabilization system as disclosed herein attached to a model and in locked orientation.

FIG. 7 illustrates the stabilization system of FIG. 6 in an unlocked orientation.

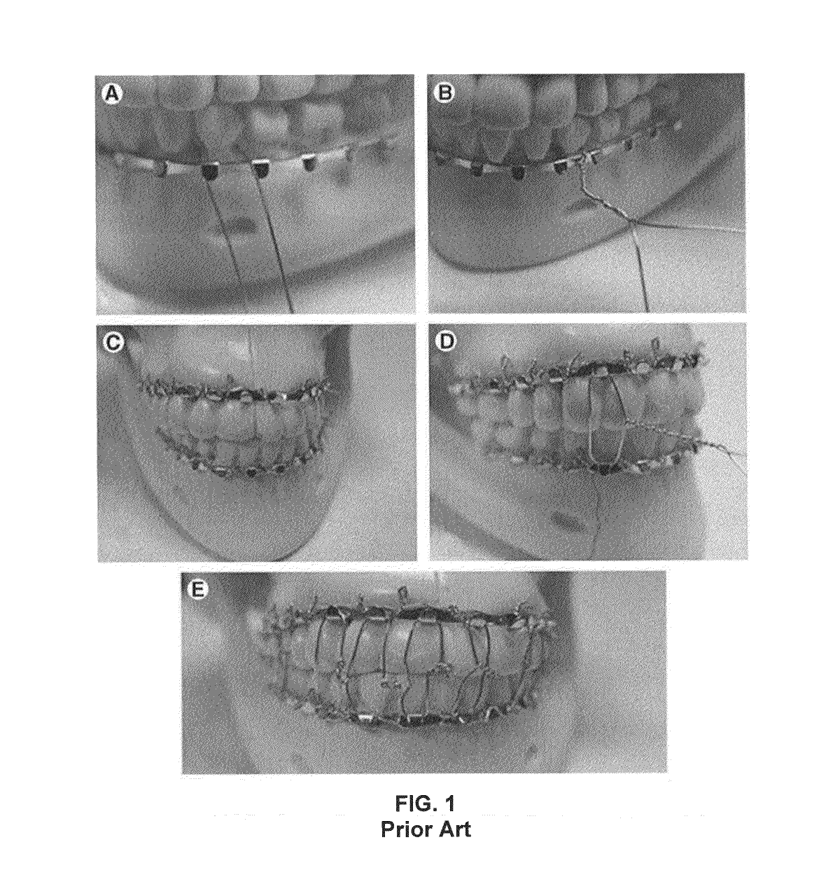

### DETAILED DESCRIPTION

Reference will now be made in detail to various embodiments of the presently disclosed subject matter, one or more examples of which are set forth below. Each embodiment is provided by way of explanation, not limitation, of the subject matter. In fact, it will be apparent to those skilled in the art that various modifications and variations may be made to the present disclosure without departing from the scope or spirit of the disclosure, For instance, features illustrated or described as part of one embodiment may be used in another embodiment to yield a still further embodiment. Thus, it is intended that the present disclosure cover such modifications and variations as come within the scope of the appended claims and their equivalents.

The present disclosure is generally directed to a mandibulomaxillary fracture stabilization system, methods of forming the system, and methods of using the system. The system includes upper and lower splints that can be custom molded to fit a patient and a locking mechanism that can temporarily lock the splints to one another.

The system provides improved safety for both the patient and medical personnel both during and after installation, For instance, the smooth lines and soft edges of the system components can help to reduce safety risks of the fixation/stabilization process as well as the recovery period. In addition, the locking mechanism can include a quick release capability, which can allow a patient to unlock the system and regain the ability for separation of the upper and lower jaw in seconds, which can provide added safety assurance in case nausea, choking, or other emergencies that may be experienced during recovery.

The system is also robust, and can safely and securely fix the jaw in place for the necessary recovery period, for instance over the course of a 6 weeks post-operative recovery period.

Beneficially, the system can be quickly installed to a patient's jaw during a procedure. For instance, system installation can be carried out in a time period that can be reduced by about 75% as compared to the time necessary to install previously known arch bar systems. The system is thus quite cost effective—meeting all clinical requirements while potentially saving thousands of dollars in operating room time.

As mentioned above, the mandibulomaxillary fixation system includes an upper and lower splint and a locking mechanism. FIG. 3 illustrates one embodiment of a splint. As can be seen, the splint is designed to extend laterally from the first side 12 (e.g., the left side) to the second side 14 (e.g., the right side) of the jaw. In the case of the lower splint, the splint can be formed so as to extend around the jaw from the left side to the right side of the mandible. In the case of the upper splint, the splint can be formed so as to extend around the jaw from the left maxilla to the right maxilla.

The splints can include tooth impressions so as to cover at least one of the posterior teeth on either side of each splint upon installation and thus be formed to snugly fit over at least a portion of the teeth. For instance, a splint can include two or more tooth impressions on either side of the splint and cover at least two or more teeth of the patient upon installation so as to provide a secure attachment as near as possible to the temporomandibular joint. In the illustrated embodiment of FIG. 3, the splint includes 5 tooth impressions (1, 2, 3, 4, 5) on the first half 15 of the splint and 5 tooth impressions (6, 7, 8, 9, 10) tooth impressions on the second half 16 of the splint. For example, the splint can include tooth impressions to cover all available molars and bicuspids of a patient upon installation. In another embodiment, a splint can include tooth impressions along the entire length of the splint and be designed to cover all of the patient's teeth upon installation.

In the illustrated embodiment, the first half 15 and the second half 16 of the splint are connected to one another with a strip 17 that forms to the front teeth but does not cover the teeth. This can improve comfort of the system by leaving the top and back of the front teeth uncovered. The connecting strap 17 is not required, however, and in other embodiments, the first half and the second half of an upper or lower splint can be separate pieces that are not physically connected to one another. In this embodiment, the two halves of the splints can be separately attached to the patient's posterior teeth.

To ensure a comfortable fit for the duration of the recovery period, the splints can be custom molded to the natural dentition of each patient. In one embodiment, a custom molded splint can be formed by use of a mold formed of the patient's jaw prior to the surgery. A mold can be formed according to any suitable formation method. For instance a standard direct-casting method can be utilized as is generally known using a suitable impression material such as polyvinyl siloxane and a standard or conventional dental impression tray.

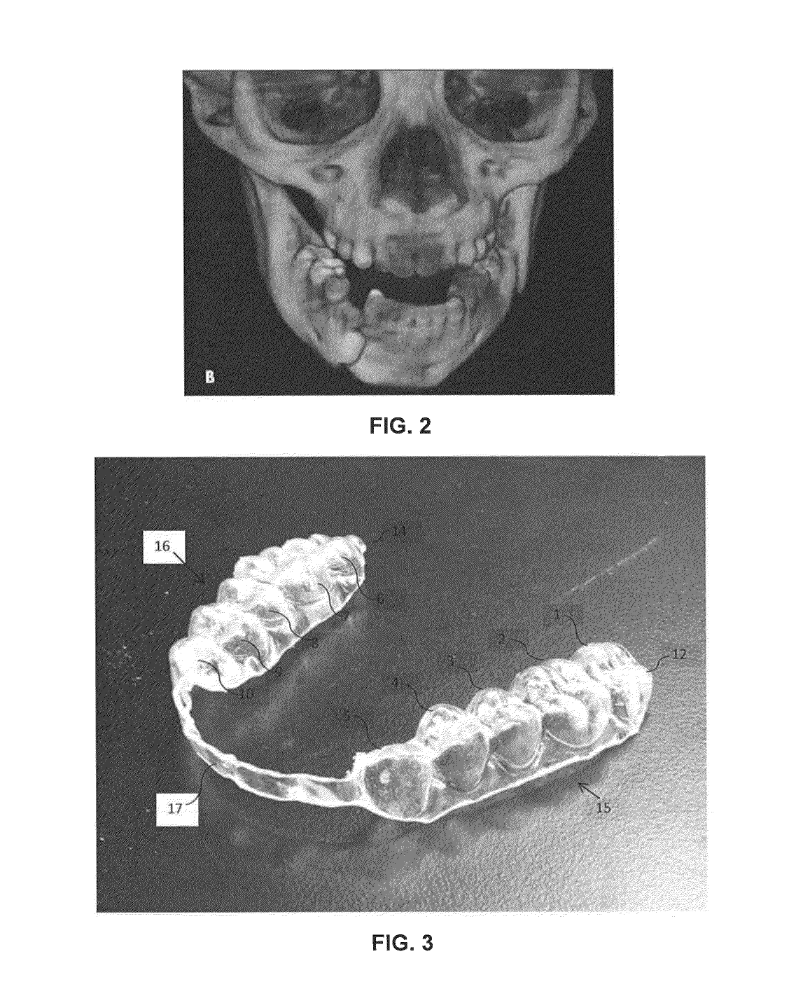

In one embodiment, a mold can be formed by use of a computer enhanced reconstruction method such as a CT reconstruction method or by use of an intraoral scanner as is known in the art A computer enhanced formation method may be particularly beneficial in applications involving traumatic accident in which it may not be possible to obtain a pre-accident mold of the patient's jaw. By way of example, FIG. 2 is a CT scan image of a fractured mandible. By use of known software, the CT image can be utilized to reconstruct an in silico model of the properly aligned jaw.

Irregardless of the method used to form a jaw model, the in silico model of the jaw can then be used to form a custom positive mold of the patient's jaw, for instance by use of 3D-printing methodology, computer aided design/computer aided manufacturing (cad/cam) methodology, etc. 3D printing of dental appliances has been described, for instance by Sun, et al. in U.S. Patent Application Publication No. 2014/0131908, Durbin, et al. in U.S. Patent Application Publication No. 2011/0171604, and Dierkes et al. in U.S. Patent Application Publication No. 2006/0131770, all of which are incorporated herein by reference.

Once formed, the positive mold can be used to form the splint. For example, a splint can be thermoformed according to a method similar to that described by Schwartz, et al. in U.S. Pat. No. 5,692,894, which is incorporated herein by reference. Briefly, a sheet or plate comprising a suitable biocompatible thermoplastic material can be pressed against the positive mold under heat and/or pressure so as to be formed to the desired shape. Vacuum formation methods as are known in the art may also be utilized. In general, the upper and lower splints will be formed of the same material, but that is not a requirement of the systems.

A conventional pressure or vacuum molding machine may be used to produce the splints from a suitable material, such as thermoformable dental material, available from Tru-Tain® Plastics, Rochester, Minn. Suitable polymers include, for example, polyesters, polycarbonates, polyvinyl chlorides, etc. Exemplary pressure molding equipment is available under the trade name BIOSTAR® from Great Lakes Orthodontics, Ltd., Tonawanda, N.Y. Exemplary vacuum molding machines are available from Raintree Essix®, Inc. A molding machine can produce the splints directly from the positive jaw mold and the formable thermoplastic material.

In another embodiment, the in silico model of the patient's jaw can be used to design an in silico splint using, e.g., an appropriate “auto-fit” or other suitable software program. The actual splints can then be directly formed by 3-D printing, cad/cam manufacturing, or the like from the in silico splint model. This method may be beneficial in some embodiments, as it can save the costs associated with the formation of a physical model of the patient's jaw. Of course, in such an embodiment, the materials as may be used to form the splints are not limited to thermoformable polymeric materials, and any suitable material may be utilized that may be printed, milled, machined, etc. to the desired shape.

Final shaping of the system components can be carried out as necessary similar to that known for other dental appliances. For instance, an appropriate polishing bur or other suitable bur can be utilized to finish the splints and remove any edges or other areas that could interfere with the fit and/or cause irritation to the patient. For instance a rotary hand-held instrument as is known in the art can be utilized or a non-rotary instrument or device (e.g., sand paper) can be utilized to finish a component of the system.

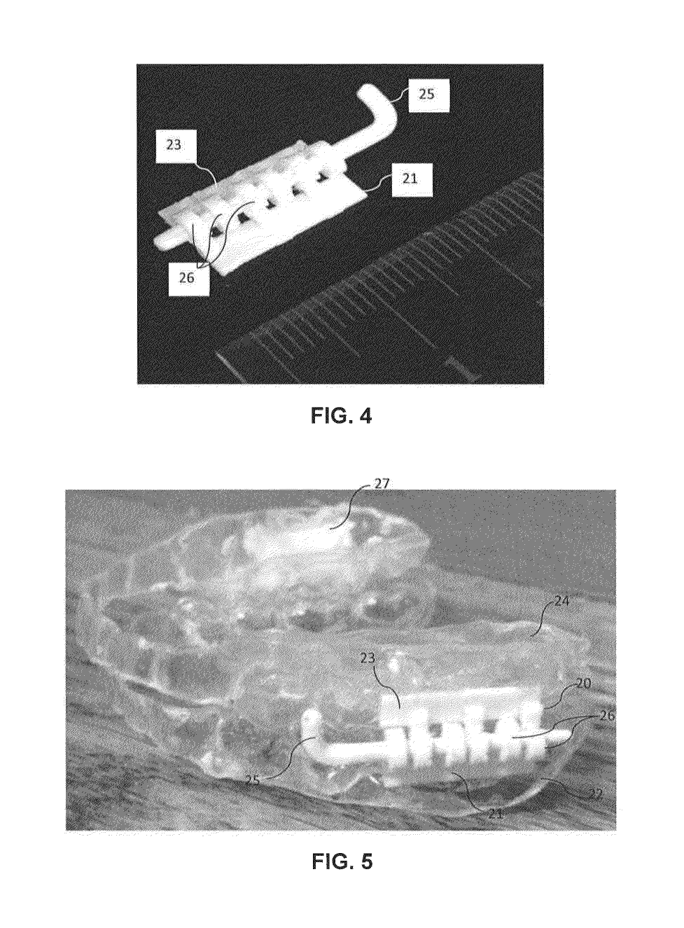

The fixation system also includes a locking mechanism that can removably lock the upper and lower splints to one another. FIG. 4 illustrates one embodiment of a locking mechanism. As can be seen, the locking mechanism has a pin and hinge design and includes a first hinge component 21, a second hinge component 23, and a pin 25. The first and second hinge components include loops 26 that, when properly aligned interlock and are held together with the pin 25, as shown. The loops 26 can be closed rings or open loops that are not completely closed in circumference.

The components of the locking mechanism can be formed of any suitable biocompatible material including, without limitation, polymeric materials (e.g., polyvinyl chloride, polycarbonate, etc.), metals (e.g., stainless steel), ceramics, etc. For example, the components of the locking mechanism can be injection molded from a suitable polyester copolymer. As the locking mechanism will be in contact with the soft tissue of the patient, the mechanism can be formed without sharp edges or rough surfaces, so as to improve comfort and prevent abrasion. When formed of a polymeric material, the material can be the same or different as the material used to form the splints.

In one embodiment, the locking mechanism can be formed in conjunction with one or both splints and can be formed from the same or a different polymeric composition, For instance, the system components including the first and second splint and the locking mechanism can be formed as a monolithic device in a 3-D printing process or other suitable process, for instance based upon an in silico model formed by use of computerized methodology as described above. Alternatively, a portion of the system components can be formed as a monolithic unit. For instance, one of the splints can be formed in conjunction with the locking mechanism and the other splint can be formed as a separate component.

The locking mechanism is not particularly limited to the hinge/pin embodiment of the figures, and other interlocking mechanisms are encompassed herein. For instance, a locking mechanism can include a first component and a second component that fit together in a nested arrangement, e.g., a male/female fitting. The locking mechanism can include a quick release fitting such as a locking tab on the inner nested component that extends through the wall of the outer nested component when locked and can be released by pressure on the tab. In any suitable arrangement, however, the locking mechanism can include a quick-release capability to as to allow the jaw to be unlocked quickly by the patient or a caretaker in case of an emergency.

As shown in FIG. 5, the locking mechanism 20 can be attached to the posterior section of the lower splint 22 and the upper splint 24. More specifically, the first component 21 can be attached to the lower splint 22 and the second component 23 can be attached to the upper splint 23. The pin 25 can be threaded through the interlocked loops 26 of the hinge to lock the mechanism and hold the splints in a fixed orientation to one another. To firmly fix the jaw in place, the system will generally include a second locking mechanism 27 that can be attached to the opposite side of the splints, as shown.

The locking mechanism can be attached to the splints by use of standard dental adhesive or any other suitable mechanism. For instance, in one embodiment, the first and second components of the locking mechanism can be heat bonded to the upper and lower splints, respectively, in conjunction with the splint formation process. In general, however, the locking mechanism can be adhered to the splints following attachment of the splints to the dentition of the patient, so as to ensure proper alignment of the mechanism components.

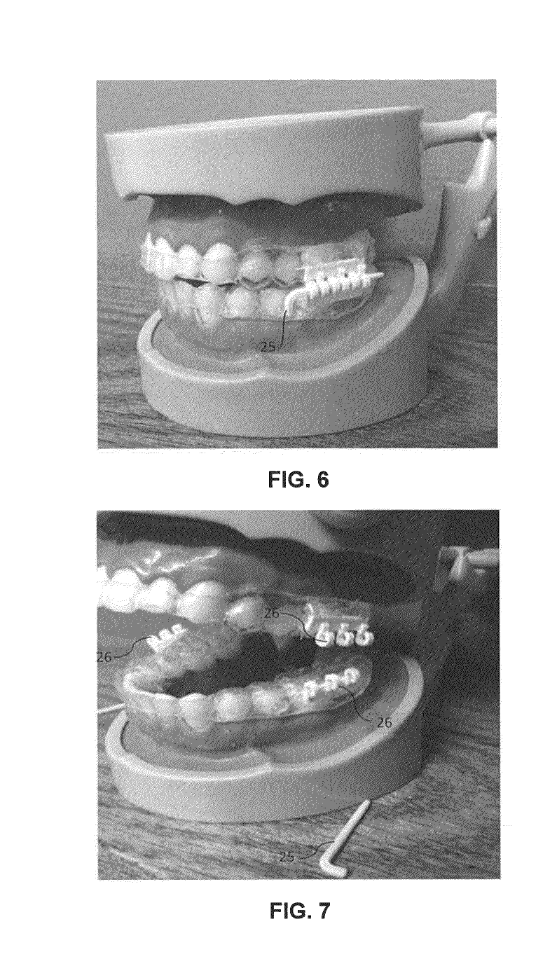

FIG. 6 and FIG. 7 illustrate the fixation system following application to a model including the system in a locked orientation (FIG. 6) and in an open, unlocked orientation (FIG. 7). As discussed above, to unlock the system, a patient or caretaker need only pull the pin 25 out of the interlocking loops 26 and the jaw can open.

To utilize and install the fixation system, the jaw can be manipulated by the care giver to fit into the pressure-formed upper and lower splints and the splints can be adhered to the dentition using suitable dental cement. A similar cement can be used to attach the first and second components of a locking mechanism to the posterior portion of the upper and lower splint, respectively. A second locking mechanism can be secured to the opposite side of the splints, firmly securing the upper and lower arches. The two components of each locking mechanism can be secured together, as with the pin in the hinge/pin configuration, to fix the jaw in place.

The device attachment method is relatively simple and can be carried out in a few minutes, for instance about 10 minutes or less. The smooth polymeric components and the lack of wires in the system can reduce device-induced injury to both patients and clinicians.

A system can include both multiple-use and single-use components. Multiple use components can include, for example, software for reconstructing a fracture from a CT image, a 3D printer for forming a model, a pressure form vacuum for forming the splints, and a rotary too for final shaping of the splints.

The single-use components can include the locking mechanism, the polymeric sheets used to form the splints, and the adhesive for attachment of the splints to the teeth and the locking mechanism to the splints. In one embodiment, the single-use components can be individually sterilized, packaged, and provided as a one-time use kit.

The fixation system can be beneficially utilized by dental specialists including otolaryngologist specialists, plastic surgeons, and oral surgery specialists that can implement the device in patients requiring mandibulomaxillary fixation, for instance patients with mandible or maxillary fractures sustained from major trauma. The device can also be implemented in patients who need their jaw broken for surgical reasons; for example, to access a tumor or corrective jaw/TMJ surgery.

The present disclosure may be further understood with reference to the example, below.

### EXAMPLE

The safety and effectiveness of the design illustrated in FIG. 5 was demonstrated through standardized testing protocols and marked equivalence to multiple predicate devices—from traditional arch bars to orthodontic retainers—in an effort to model the current uses of the device's form and function.

Mechanical testing was completed to verify the resilience and stability of the device. Testing standards including ASTM F748-06 (Biological Testing), ASTM F2258-05 (Tissue Adhesion Testing), and ASTM D638-10 (Tensile Properties of Plastics) were employed during verification and validation measures. Mechanical testing was also conducted to ensure the device would hold up to the mechanical forces of the jaw.

Tensile testing of the plastic material used to form the splints was performed on an Instron® tensile testing machine. Specimens were prepared by soaking them in HBSS solution at 37° C. for 0, 1, and 4 days. This simulated the conditions the material would experience in the mouth. The material was required to withstand a force of 221N before plastic deformation, which is the reported maximum opening force achievable by the human jaw. All test specimens withstood the indicated force, thus satisfying this design requirement.

The time to install a device was quantified by both clinical and laypersons. Each subject conducted a mock installation and time necessary to complete the installation was recorded for each trial. On average, installation was completed in 6 minutes and 41 seconds (±1 Standard Deviation). As most current devices take over 45 minutes to install, the installation time for the disclosed devices was improved significantly.

The efficiency of the device's quick release locking mechanism was also tested using volunteers and a model mouth to simulate the installed device. The volunteers removed the locking pins from the two mechanisms (one on either side of the splints) as quickly as possible, and the total time was recorded. The average time to remove both pins was 5.68 seconds.

Reinsertion of the pins was also tested and found to be straightforward and simple. From a functionality standpoint, the locking mechanism has been designed to ensure that the loops of the locks are spaced apart enough so that they do not interfere with one another directly once the pin is removed.

While the present subject matter has been described in detail with respect to specific embodiments thereof, it will be appreciated that those skilled in the art, upon attaining an understanding of the foregoing may readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art.

What is claimed is:

1. A mandibulomaxillary stabilization system comprising:
an upper splint that is removably attachable to upper dentition, the upper splint comprising a first polymeric material, the upper splint comprising a left upper posterior tooth impression and a right upper posterior tooth impression;
a lower splint that is removably attachable to lower dentition, the lower splint comprising a second polymeric material, the lower splint comprising a left lower posterior tooth impression and a right lower posterior tooth impression; and
a locking mechanism, the locking mechanism including a first component that is associated with the upper splint and a second component that is associated with the lower splint, the first and second components releasably interlocking with one another.

2. The system of claim 1, the upper splint comprising at least two left upper tooth impressions and at least two right upper tooth impressions.

3. The system of claim 1, the lower splint comprising at least two left lower tooth impressions and at least two right lower tooth impressions.

4. The system of claim 1, the first and second component of the locking mechanism forming a hinge, the locking mechanism further comprising a removable pin.

5. The system of claim 1, wherein the first polymeric material and the second polymeric material are the same.

6. The system of claim 1, wherein the first polymeric material and the second polymeric material are thermoformable.

7. The system of claim 1, the locking mechanism comprising a polymeric material.

8. The system of claim 1, the locking mechanism comprising stainless steel.

9. A kit comprising the polymer material of the upper splint, the polymeric material of the lower splint, and the locking mechanism of claim 1.

10. A method for stabilizing a temporomandibular joint comprising:
adhering a first polymeric splint to the upper dentition of a subject;
adhering a second polymeric splint to the lower dentition of a subject; and
locking a first component and a second component of a locking mechanism to one another, the first component of the locking mechanism being associated with the first polymeric splint and the second component of the locking mechanism being associated with the second polymeric splint such that upon locking the components together, the joint is secured in a fixed orientation.

11. The method of claim 10, the method further comprising attaching the first component to the first polymeric splint and attaching the second component to the second polymeric splint.

12. The method of claim 10, the step of locking the first component and the second component to one another comprising sliding a pin into interlocking loops of the first and second components.

13. A method for forming a mandibulomaxillary stabilization system comprising:
forming a first splint to fit the upper dentition of a patient;
forming a second splint to fit the lower dentition of a patient;
associating a first component of a locking mechanism with the first splint; and
associating a second component of a locking mechanism with the second splint.

14. The method of claim 13, the method of forming the first and second splints comprising forming the first and second splints against a mold of the upper dentition and lower dentition, respectively, of the patient.

15. The method of claim 14, the method further comprising forming the mold of the upper dentition of the patient and forming the mold of the lower dentition of the patient.

16. The method of claim 15, wherein the molds are formed according to a 3D printing process or a computer aided design/computer aided manufacturing process.

17. The method of claim 15, wherein the molds are formed by use of a computerized tomography scan or an intraoral scan of the patient's jaw.

18. The method of claim 13, wherein the first splint and the second splint are thermoformed.

19. The method of claim 13, wherein the first splint and the second splint are vacuum formed.

20. The method of claim 13, wherein the first splint and the second splint are formed according to a 3D printing process or a computer aided design/computer aided manufacturing process.
